(19) United States
(12) Patent Application Publication
(10) Pub. No.: US 2018/0036213 A1
(43) Pub. Date: Feb. 8, 2018

(54) A PERSONAL CARE COMPOSITION

(71) Applicant: Conopco, Inc., d/b/a UNILEVER, Englewood Cliffs, NJ (US)

(72) Inventors: Yajuan LI, Shanghai (CN); Sheng MENG, Shanghai (CN); Qiang QIU, Trumbull, CT (US); Wenqing REN, Shanghai (CN); Xia ZHENG, Shanghai (CN)

(73) Assignee: Conopco, Inc., d/b/a UNILEVER, Englewood Cliffs, NJ (US)

(21) Appl. No.: 15/555,112

(22) PCT Filed: Feb. 15, 2016
(86) PCT No.: PCT/EP2016/053108

§ 371 Date: Sep. 1, 2017

(30) Foreign Application Priority Data
Mar. 10, 2015 (CN) PCT/CN2015/073930
Jun. 1, 2015 (EP) 15170101.8

(51) Int. Cl. A61K 8/19; A61K 8/06; A61K 8/27; A61Q 17/04; A61K 8/29; A61Q 19/08; A61K 8/36

(52) U.S. Cl. CPC A61K 8/19; A61Q 19/08; A61K 8/06; A61K 8/361; A61Q 17/04; A61K 8/29; A61K 8/27; A61K 2800/43

(57) ABSTRACT

The invention relates to a personal care composition that provides the excellent sensorial of a vanishing cream while ensuring sun protection and good makeup property though incorporation of pigments. The above objects are achieved by selective use of a specific range of mole ratio of soap:fatty acid in the vanishing cream base.

### FIELD OF THE INVENTION

The invention relates to a personal care composition. The invention more particularly relates to a personal care composition that provides the excellent sensorial of vanishing creams while ensuring sun protection and good make-up effectiveness from the high amounts of pigments incorporated therein.

### BACKGROUND OF THE INVENTION

Creams are a very commonly used form of cosmetic vehicle for delivery of actives for personal care benefits. Various different types of creams are available and each type of cream provides a different sensorial property. Consumers living in hot, humid climates, or consumers with oily skin, desire personal care products that have unique tactile properties during use. Specifically, such products should, upon application to the skin, deliver a high skin friction and a matte finish to overcome the oily skin feel and shiny skin appearance. The greater the increase in skin friction, the less greasy the user perceives the product to be. More specifically, the typical sensory and optical features of these high skin friction and matte finish creams, also known as vanishing creams, provide a dry, draggy, non-greasy feel to skin; provide a non-shiny, matte finish; spread easily on the skin; and absorb or “vanish” rapidly into the skin.

Traditionally, vanishing creams contain high levels of fatty acid (e.g. stearic acid or palmitic acid or a mixture of these) and alkali metal soap as an emulsifier, which is important for the physical stability of the product. The soap is formed by the in situ neutralization with caustic potash or other alkali on a portion of the fatty acid. In the past, vanishing creams are typically claimed to contain 1 to 25%, preferably 5 to 20% fatty acid and 0.1 to 10%, preferably 0.1 to 3% soap. While the above broad range of fatty acid and soap may be generally disclosed, the vanishing cream sensorial heretofore are obtained when the mole ratio of the soap to fatty acid is less than 0.22.

In addition to the cosmetic vehicle, personal care compositions usually comprise other actives which provide physical or biological protection or benefit to the skin e.g. photoprotection, skin lightening, anti-inflammatory benefits, anti-acne and other anti-microbial benefits among a host of other advantages. Photoprotection and make up/beautifying benefits are often highly sought after. Photoprotection is desired for protecting the skin from solar radiation which includes ultraviolet (UV) radiation, wavelength of which is between 200 nm and 400 nm. Exposure of skin to UV-A (320 to 400 nm) and UV-B (290 to 320 nm) causes various problems like reddening of the skin, localized irritation, sunburn, melanoma and formation of wrinkles. Therefore, it is desirable to protect the skin and other keratinous substrates of the human body from the harmful effects of both UV-A and UV-B radiation.

Photoprotection may be delivered through incorporation of organic sunscreens or inorganic sunblocks or a combination of the two. Commonly known inorganic sunblocks include titanium dioxide and/or zinc oxide which may be tailored for specific use through use of different particle sizes, surface morphologies or surface coatings or through tailored hollow sphere technologies to render the desired optical and photoprotection benefits. Beautifying/make up benefits are delivered through use of specific dyes or pigments which give the skin a desired even skin tone. Dyes are generally organic compounds and are usually water soluble which impart colour to the desired substrate. Pigments are generally inorganic compounds and are usually water insoluble and they too provide colour to the desired substrate. Due to the sensitive nature of skin against irritation and other inflammatory causing triggers, pigments are generally preferred for inclusion in personal care products for imparting make-up benefits to the skin especially on the face. Pigments come in various colours like blue, red, brown, yellow, black and even white.

The present inventors embarked upon developing a personal care composition through a vanishing cream base which would provide good photoprotection and excellent make up property through use of pigments. WO2015/018606A1 (Unilever) discloses a cosmetic composition comprising fatty acid, fatty acid salt, and pigment, wherein the cation of fatty acid salt comprises inorganic cation and organic ammonium, and the total amount of fatty acid and fatty acid salt is at least 5% by weight of the composition.

The present inventors found that when high amounts of such pigments or inorganic particles were included in the traditional vanishing creams the above mentioned sensorial properties are compromised. Using all the teachings of published information they were not able to solve the above problem. So, through extensive experiments they came up with the inventive understanding that this problem can be solved by manipulating the mole ratio of the soap to fatty acid, at higher values in order to not only give the desired photoprotection and make-up benefits but also have the consumer desired vanishing cream base sensorial. Another problem faced by the inventors was the instability of the composition in terms of sedimentation of the inorganic particles in the traditional vanishing creams. Surprisingly the solution in terms of the inventive mole ratio ranges also solved the instability problem thereby providing a personal care composition that delivers the desired consumer benefits while not compromising on the product stability and tactile sensorial on topical application of the product. By high amounts of such pigments or inorganic particles is meant that the total amount of these particulate matter is in the range of 2 to 8%, preferably 3 to 6%. It was observed that compositions having particulate matter less than the above amounts generally do not face such sensorial or instability problems.

It is thus an object of the present invention to obviate the drawbacks of the prior art and provide a stable vanishing cream composition where high amounts of inorganic particulates can be incorporated.

Another object of the present invention is to achieve the above object while ensuring that the desired vanishing cream sensorial is not compromised.

It is yet another object of the present invention to achieve both of the above objects while obtaining the storage stability of the composition in terms of even suspension of the particulates over long period of time.

### SUMMARY OF THE INVENTION

The present invention provides for a personal care composition comprising:
(a) 6 to 17% by weight of a C12 to C20 fatty acid;
(b) 2 to 6% by weight of a C12 to C20 fatty acid soap;
(c) 2 to 8% by weight of a white pigment; and
(d) a cosmetically acceptable carrier;
   wherein the mole ratio of the soap to fatty acid is from 0.23 to 0.5.

It is preferred that the composition further comprises a coloured pigment.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y.

When for a specific feature multiple preferred ranges are described in the format “from x to y”, it is understood that all ranges combining the different endpoints are also contemplated.

By a personal care composition as used herein, is meant to include a composition for topical application especially to the sun-exposed areas of the skin of mammals, especially humans. Such a composition may be generally classified as leave-on or rinse off, and includes any product applied to a human body for also improving appearance, cleansing, odor control or general aesthetics. It is more preferably a leave-on product. The composition of the present invention is in the form of a cream which may be applied to the desired skin surface using ones fingers or hands or applied with an implement or via a face mask, pad or patch. Non-limiting examples of such sunscreen compositions include leave-on skin creams, antiperspirants, and deodorants. “Skin” as used herein is meant to include skin on the face and body (e.g., neck, chest, back, arms, underarms, hands, legs, buttocks and scalp) and especially to the sun exposed parts thereof.

By a white pigment as per this invention is meant an inorganic particulate material which is visibly white in colour.

By a coloured pigment as per this invention is meant an inorganic particulate material which is not visibly white in colour. Coloured pigment as per this invention is preferably an inorganic particulate material which is visibly black, brown, pink, orange, red, yellow or blue in colour, or a combination of these colours.

By way of the present invention, the consumer is delivered the benefit of the excellent sensorial of a vanishing cream through which skin may be moisturised and given an instant even tone as well as photoprotection. Additionally, by way of appropriate benefit agents, skin lightening may be delivered to the consumer.

The composition of the invention comprises a fatty acid and a soap which are the key ingredients of a vanishing cream base. C₁₂ to C₂₀ fatty acids are present in 6 to 17%, preferably 8 to 15% by total weight of the composition. When the composition is being prepared, higher amounts of fatty acid may be added but a percentage of this is in-situ neutralised through use of an alkali metal hydroxide. When the neutralisation reaction is complete, it is expected that the amount of fatty acid remaining (un-neutralised) is in 6 to 17% by total weight of the composition. The fatty acids may be saturated or unsaturated fatty acids. While C₁₂ to C₂₀ fatty acids may be used, it preferred that C₁₄ to C₁₈ fatty acids are used. More preferably, the fatty acid is stearic acid or palmitic acid or a mixture of the two. The fatty acid in the present invention is preferably prepared using hystric acid which is substantially (generally about 90 to 95%) a mixture of stearic acid and palmitic acid in a ratio of between 55:45 to 45:55. Soap in the present invention is generally prepared by in-situ neutralisation of the fatty acid stock added to the reaction mixture. Thus, it is preferred that the soap has a carbon chain length which corresponds to the chain length of the free fatty acid in the composition. Soap is present in 2 to 6%, preferably 3 to 6% by total weight of the composition. The soap is formed from the fatty acid through use of alkali metal hydroxides like sodium or potassium hydroxide. Of the two, potassium hydroxide is more preferred. Thus the soap is preferably a potassium soap (potassium salt of fatty acid).

By the term ‘mole ratio of soap to fatty acid’ is meant mole of soap per mole of fatty acid remaining in the composition. Thus, if 1 mole of fatty acid is taken and neutralized with an alkali to form 0.2 moles of soap and if 0.8 moles of fatty acid is remaining in the composition, then the mole ratio of soap to fatty acid is 0.2/0.8=0.25. It is important that the mole ratio of the soap to the fatty acid in the composition after the neutralisation reaction is complete is in the range of 0.23 to 0.5, preferably 0.25 to 0.42. Generally, traditional vanishing creams have the mole ratio of soap to fatty acid less the 0.23. Use of these higher mole ratios have advantageously been used in the present invention to surprisingly provide the benefits summarised hereinabove especially when the composition comprises inorganic white pigments like titanium dioxide and in a preferred aspect inorganic coloured pigments. When the ratio is expressed as a weight ratio of soap to fatty acid, the composition preferably has a weight ratio of soap to fatty acid in the range of 0.26 to 0.55 preferably 0.28 to 0.45. Thus, the extent of neutralization of the fatty acid added into the reaction mixture, in the present invention, is at least 19% and could be up to 33% but is preferably up to 30%, while traditional vanishing creams have been prepared with the extent of neutralisation of less than 19%, often less than 18%. To achieve the extent of neutralisation, in the present invention, the stock of fatty acid mixture in the reaction vessel is dosed with alkali metal hydroxide of from 0.4 to 1%, preferably 0.5 to 0.7% by weight of the composition.

The composition of the invention comprises 2 to 8% white pigment. Traditionally, such inorganic particulates are included in 0.1 to at most 2%, often 0.1 to 1%. The inclusion of high amounts of white pigments as in the present invention makes it difficult to meet the desired sensorial of a vanishing cream while ensuring even dispersion of the particles in the composition at the manufacturing and transit conditions and during storage on the retail and consumer shelf. The above problem has been solved by way of the selective mole ratio defined above while ensuring that the benefit agents included therein perform as they are expected in their native state. The composition of the invention comprises 2 to 8% white pigment which generally have a refractive index higher than 1.8. The refractive index of the white pigment may be as high as 3.0. Preferred range of refractive index of the white pigment is from 1.8 to 2.2. Suitable white pigment which may be included as per the above criterion are zinc oxide, or titanium dioxide. More preferred white pigment is titanium dioxide. The amount of white pigment that is incorporated in the composition is preferably 3 to 6%, more preferably 4 to 6% by weight of the composition. The white pigment preferably has an average particle size in the range of 100 nm to 1 micrometer, more preferably from 300 nm to 1 micrometer. The white pigments may preferably be hydrophobically coated. Suitable hydrophobic coating materials are aluminium stearate, silicones or ferric stearate.

The composition of the invention preferably comprises a coloured pigment. A coloured pigment is generally an inorganic particulate material that is visibly coloured e.g. having a red, pink, brown, orange, yellow, blue or black colour. The coloured pigment as per the present invention preferably is red, yellow or black or a combination of these, most preferably a combination of all three of these coloured materials. Most preferred coloured pigment as per the present invention is iron oxide. The coloured pigment is preferably present in 0.1 to 2%, more preferably 0.5 to 1.5% by weight of the composition. Of the various colours, the yellow pigment preferably predominates and is preferably present in 0.05 to 1.2%, while the red pigment may be present in 0.01 to 0.3%, while the black pigment may be present in 0.01 to 0.2% by weight of the composition. The coloured pigment may also optionally be coated. Suitable coating materials are fatty acid or fatty acid salts, preferably stearic acid, polyhydroxystearic acid or a stearate. The coloured pigment preferably has an average particle size in the range of 100 nm to 1 micrometer, more preferably from 300 nm to 1 micrometer.

In addition to the pigments, the composition may comprise inorganic sunblocks. Inorganic sunblocks that may be incorporated include titanium dioxide or zin oxide, the particle size of which is generally below 100 nm in primary particle size. It is preferred that the total inorganic particles in the composition of the invention is less than 10 wt %, preferably less than 8 wt %. The composition of the invention preferably comprises one or more organic sunscreens. The organic sunscreens are preferably chosen from the following eight major groups: (1) benzophenones, (2) anthranilates, (3) dibenzoylmethanes (4) salicylates, (5) cinnamates, (6) camphores (7) p-amino benzoic acid (PABA), (8) diphenl acrylic acid or their derivatives or mixtures.

The organic sunscreens may be of the UV-A or of the UV-B sunscreen types. Preferred UV-A sunscreen is a dibenzoylmethane, triazine, triazone, or benzophenone derivative. A more preferred UV-A sunscreen belongs to the dibenzoylmethane group. When present, this is included in 0.1 to 3%, more preferably 0.1 to 2%, most preferably 0.2 to 1.5% dibenzoylmethane or its derivative. Preferred dibenzoylmethane derivatives is 4-tert.-butyl-4′-methoxydibenzoylmethane.

The composition of the invention may comprise a UV-B sunscreen. UV-B organic sunscreen is preferably selected from the class of cinnamic acid, salicylic acid, diphenyl acrylic acid or derivatives thereof. A few of the preferred UV-B sunscreens which are commercially available and useful for inclusion in the composition of the invention are Octisalate™, Homosalate™, NeoHelipan™, Octocrylene™, Oxybenzone™ or Parsol MCX™. The UV-B organic sunscreen when included in the composition of the invention preferably belongs to the cinnamic acid compound group. It is preferably 2-ethyl-hexyl-4-methoxy cinnamate which is commercially available as Parsol MCX. When present, UVB sunscreen is included in 0.1 to 4%, preferably from 0.1 to 3%, more preferably 0.2 to 2% by weight of the composition. Most preferably the composition comprises both a UV-A sunscreen and a UV-B sunscreen. The composition of the invention has the additional advantage that it provides an in-vivo SPF (sun protection factor) of at least 15.

The composition of the invention may additionally comprise a skin lightening agent. The skin lightening agent is preferably chosen from a vitamin B3 compound or its derivative e.g. niacin, nicotinic acid, niacinamide or other well known skin lightening agents e.g. aloe extract, ammonium lactate, azelaic acid, kojic acid, citrate esters, ellagic acid, glycolic acid, green tea extract, hydroquinone, lemon extract, linoleic acid, magnesium ascorbyl phosphate, vitamins like vitamin B6, vitamin B12, vitamin C, vitamin A, a dicarboxylic acid, resorcinol derivatives, hydroxycarboxylic acid like lactic acid and their salts e.g. sodium lactate, and mixtures thereof. Vitamin B3 compound or its derivative e.g. niacin, nicotinic acid, niacinamide are the more preferred skin lightening agent as per the invention, most preferred being niacinamide. Niacinamide, when used, is preferably present in an amount in the range of 0.1 to 10%, more preferably 0.2 to 5%, and most preferably 0.5 to 2% by weight of the composition.

The composition comprises a cosmetically acceptable carrier. A suitable cosmetically acceptable carrier is water or an aqueous solution comprising a thickener, on oil phase/solvent, a co-surfactant and one or more cosmetically acceptable ingredient. Cosmetically acceptable carrier may be included in 20 to 90% by weight of the composition.

The composition preferably includes water. Water is preferably included in 35 to 90%, more preferably 50 to 85%, further more preferably 50 to 80% by weight of the composition.

The composition of the invention preferably comprises a co-surfactant which is preferably a fatty alcohol more preferably a cetyl alcohol. It is preferably included in 0.1 to 3%, preferably 0.5 to 2% by total weight of the composition.

The composition of the invention preferably comprises a thickener which may be a polymer selected from the class of acrylate/R-methacrylate copolymer or crosspolymer, or an acrylate/R-alkyl acrylate crosspolymer; or a polymer selected from the class of copolymers of acryloyldimethyltaurate with vinyl pyrrolidone, methacrylate or R-alkyl acrylate. The polymer thickener is preferably acrylates/steareth-20 methacrylate copolymer, ammonium acryloyl dimethyl taurate/beheneth-25 methacrylate or a mixture thereof. Such thickeners may be included in 0.01 to 0.5% more preferably from 0.05 to 0.3% by weight of the composition.

Other cosmetically acceptable ingredients which are usually included in such personal care compositions are humectants, emollients and skin soothing and healing agents. Suitable humectants include polyhydric alcohols e.g. glycerine, polyethylene glycols and propylene glycols which are advantageously included in the composition of the present invention. Of these glycerine is especially preferred. Humectants when included are preferred in the range of 0.1 to 5%, more preferably in the range of 0.5 to 3% by total weight of the composition. Emollients are also a class of highly preferred ingredients in such personal care compositions which may be selected form silicones, and fatty acid esters. Especially preferred emollients which may be included are dimethicones, isopropyl myristate and cetearyl ethylhexanoate. Emollients may be included in 0.5 to 5%, preferably 1 to 3% by weight of the composition. Other useful skin beneficient agents which sooth and heal which may be included are allantoin, aloe vera extracts, tocopheryl aceate, pyridoxine hydrochloride, sodium ascorbyl phosphate and 12-hydroxystearic acid or combinations thereof.

The cosmetically acceptable carrier according to the invention may also comprise other diluents. The diluents act as a dispersant or carrier for other materials present in the composition, so as to facilitate their distribution when the composition is applied to the skin. Diluents other than water can be other liquids like solvents or could be powders.

The compositions of the present invention can comprise a wide range of other optional components. The CTFA Cosmetic Ingredient Handbook, Second Edition, 1992, which is incorporated by reference herein in its entirety, describes a wide variety of non-limiting cosmetic and pharmaceutical ingredients commonly used in the skin care industry, which are suitable for use in the compositions of the present invention. Examples include: antioxidants, binders, biological additives, buffering agents, colorants, astringents, fragrance, opacifying agents, conditioners, exfoliating agents, pH adjusters, preservatives, natural extracts, essential oils, and skin sensates.

The invention will now be illustrated with the help of the following non-limiting examples.

### EXAMPLES

### Example 1 to 9: Samples at Various Extents of Neutralisation (Mole Ratio of Soap:Fatty Acids)

Various compositions were prepared by mixing the ingredients shown in Table-1 at a temperature in the range of 70 to 80° C., that ensured that all of the potassium hydroxide was consumed in neutralising part of the fatty acid to soap. The compositions (vanishing creams) were tested for stability using the following procedure: Stability, as used herein, refers to the ability of the composition to maintain its appearance and macroscopic structure without phase separation when stored under the following conditions.

Samples were stored at 4° C., 25° C., 37° C., and 45° C. for three months or 50° C. for one month. For stability measurement, samples were checked at the end of week 1, week 2, week 4, week 8 and week 12. The appearance of the samples were observed at the end of 12 weeks and left at 25° C. for 24 hours before another observation was taken. The results are reported in Table 1.

TABLE 1

| | Examples (wt %) | | | | | | | | |
|---|---|---|---|---|---|---|---|---|---|
| Ingredient | 1 | 2 | 3 | 4 | 5 | 6 | 7 | 8 | 9 |
| Hystric | 17.0 | 12.0 | 12.0 | 12.0 | 12.0 | 12.0 | 10.0 | 15.0 | 17.0 |
| acid^{(a)} | | | | | | | | | |
| KOH | 0.49 | 0.36 | 0.46 | 0.55 | 0.55 | 0.64 | 0.46 | 0.68 | 0.77 |
| TiO₂^{(b)} | 5.00 | 5.00 | 5.00 | 5.00 | 3.00 | 5.00 | 5.00 | 5.00 | 5.00 |
| Pigment^{(c)} | 1.20 | 1.20 | 1.20 | 1.20 | 0.97 | 1.20 | 1.20 | 1.20 | 1.20 |
| Glycerine | 1.00 | 1.00 | 1.00 | 1.00 | 1.00 | 1.00 | 1.00 | 1.00 | 1.00 |
| Cetyl | 1.50 | 1.50 | 1.50 | 1.50 | 1.50 | 1.50 | 1.50 | 1.50 | 1.50 |
| alcohol | | | | | | | | | |
| Parsol | 0.75 | 0.75 | 0.75 | 0.75 | 0.75 | 0.75 | 0.75 | 0.75 | 0.75 |
| MCX^{(d)} | | | | | | | | | |
| Parsol | 0.40 | 0.40 | 0.40 | 0.40 | 0.40 | 0.40 | 0.40 | 0.40 | 0.40 |
| 1789^{(e)} | | | | | | | | | |
| Niacinamide | 1.25 | 1.25 | 1.25 | 1.25 | 1.25 | 1.25 | 1.25 | 1.25 | 1.25 |
| Thickener^{(f)} | 0.15 | 0.15 | 0.15 | 0.15 | 0.15 | 0.15 | 0.15 | 0.15 | 0.15 |
| Water | To 100 | To 100 | To 100 | To 100 | To 100 | To 100 | To 100 | To 100 | To 100 |
| Extent of | 13.70 | 14.50 | 18.20 | 21.80 | 21.80 | 25.50 | 21.80 | 21.80 | 21.80 |
| neutraliation | | | | | | | | | |
| (%) | | | | | | | | | |
| Mole ratio^{(g)} | 0.16 | 0.17 | 0.226 | 0.28 | 0.28 | 0.34 | 0.29 | 0.28 | 0.28 |
| Stability | Poor | Poor | Poor | Good | Good | Good | Good | Good | Good |

^{(a)}Hystric acid is a 55:45 mixture of palmitic acid and stearic acid
^{(b)}Commercial sample of TiO₂ was purchased from Kobo under the tradename INBP70U
^{(c)}Pigment used was a mixture of various iron oxides comprising R:Y:B (red:yellow:black) commercially available from Kobo under the tradenames INBP75ER; INBP55EY; INBP75EB in a weight ratio of 1:8.4:0.8
^{(d)}Parsol MCX is UV-B sunscreen Octyl methoxy cinnamate
^{(e)}Parsol 1789 is UV-A sunscreen Butyl methoxydibenzoyl methane
^{(f)}The thickener used was Ammonium Acryloyldimethyltaurate/Beheneth-25 Methacrylate crosspolymer
^{(g)}Mole ratio indicates the mole ratio of soap:fatty acid

The data in Table-1 indicates that Examples outside the invention (Examples 1 to 3) where the mole ratio of soap:fatty acid was less than 0.23) exhibited poor stability in that the samples showed phase separation. The examples as per the invention (Examples 4 to 9) where the mole ratio of soap:fatty acid is higher than 0.23 demonstrated good stability. Further, samples of Examples 4 to 9 exhibited good vanishing cream sensory when rubbed on to skin.

### Example 4 and A: Results of a Consumer Trial with a Composition as Per the Invention as Compared to a Market Sample

### Example 4: A Composition as Per Example-4 (Table-1) was Used in the Consumer Trial

Example A: is a popular skin care cream available in the market which (from the back of pack information) is known to contain titanium dioxide (white pigment), iron oxides (coloured pigment), niacinamide (skin lightening agent), and thickeners and emollients that in combination is claimed to give fairer looking skin, an even tone of the skin with instant benefits.

The above two samples were tested in a consumer trial with about 200 consumers in the city of Mumbai, India. 109 users used sample of Example-4 and another (separate) group of 100 consumers used sample of Example A. The consumers gave their feedback on the attributes (see Table-2) of the respective products after they used the creams, once a day, for 14 days.

The consumers then rated the products that they used on various attributes and the summary of their preference is shown in Table-2 below. The numbers in the Table-2 is the percentage of people, who used the product, who agreed with the attribute indicated.

TABLE 2

| | | Example |
|---|---|---|
| Attribute | Example 4 | A |
| Non-oiliness while applying the cream | 100 | 92 |
| Non-oiliness after applying the cream | 99 | 94 |
| Non-stickiness while applying the cream | 100 | 94 |
| Non-stickiness after applying the cream | 98 | 92 |
| Makes my skin fairer/lighter/whiter | 91 | 83 |
| Instantly reduces the appearance of dark spots and | 87 | 75 |
| blemishes | | |
| Instantly reduces the appearance of dark circles | 82 | 71 |
| Reduces blemishes/marks/dark spots | 93 | 79 |
| Gives me better instant fairness/brightness vs. my | 92 | 82 |
| regular cream | | |

The data in Table-2 above indicates that composition as per the present invention (Example-4) is vastly superior in terms of sensorial properties as well as functional skin lightening benefits, as is evident from the percentage of consumers who prefer the respective attributes.

The present invention thus provides a stable vanishing cream composition where high amounts of inorganic particulates can be incorporated while ensuring the desired vanishing cream sensorial. Additionally the composition delivers the desired skin benefit agents sufficiently well to be consumer perceivable.

1. A personal care composition comprising:
a) 6 to 17% by weight of a C₁₂ to C₂₀ fatty acid;
b) 2 to 6% by weight of a C₁₂ to C₂₀ fatty acid soap;
c) 2 to 8% by weight of a white pigment; and
d) a cosmetically acceptable carrier;
wherein the mole ratio of the soap to fatty acid is from 0.23 to 0.5.

2. The composition according to claim 1 wherein the fatty acid is present in an amount from 8 to 15% by weight of the composition.

3. The composition according to claim 1 wherein the fatty acid is stearic acid, palmitic acid or a mixture thereof.

4. The composition according to claim 1 wherein the soap is present in an amount from 3 to 6% by weight of the composition.

5. The composition according to claim 1 wherein the soap is a sodium or potassium salt of fatty acid.

6. The composition according to claim 1 wherein the mole ratio of soap to fatty acid is from 0.25 to 0.42.

7. The composition according to claim 1 wherein the white pigment is titanium dioxide or zinc oxide.

8. The composition according to claim 1 wherein the white pigment is present in an amount from 3 to 6% by weight of the composition.

9. The composition according to claim 1 wherein the composition further comprises a coloured pigment.

10. The composition according to claim 9 wherein the coloured pigment is iron oxide.

11. The composition according to claim 9 wherein the coloured pigment is present in an amount from 0.1 to 2% by weight of the composition.

12. The composition according to claim 1 wherein the composition further comprises co-surfactant.

13. The composition according to claim 1 wherein the composition further comprises a polymer selected from the class of acrylate/R-methacrylate copolymer or crosspolymer, or an acrylate/R-alkyl acrylate crosspolymer; or a polymer selected from the class of copolymers of acryloyldimethyltaurate with vinyl pyrrolidone, methacrylate or R-alkyl acrylate.

14. The composition according to claim 13 wherein the polymer is acrylates/steareth-20 methacrylate copolymer, ammonium acryloyl dimethyl taurate/beheneth-25 methacrylate or a mixture thereof.

15. The composition according to claim 1, wherein the composition is a vanishing cream.

16. The composition according to claim 12 wherein the co-surfactant is cetyl alcohol.
